(19) United States
(12) Patent Application Publication
(10) Pub. No.: US 2021/0186398 A1
(43) Pub. Date: Jun. 24, 2021

(54) METHOD AND APPARATUS FOR TESTING PERSONALITY BASED ON ANIMAL SYMBOLS

(71) Applicant: Kwang Hyun CHOI, Yongin-si (KR)

(72) Inventor: Kwang Hyun CHOI, Yongin-si (KR)

(21) Appl. No.: 17/127,950

(22) Filed: Dec. 18, 2020

(30) Foreign Application Priority Data
Dec. 20, 2019 (KR) 10-2019-0171625
Apr. 16, 2020 (KR) 10-2020-0046203

(51) Int. Cl. A61B 5/16; A61B 5/00

(52) U.S. Cl. CPC A61B 5/167; A61B 5/7435; A61B 5/165

(57) ABSTRACT

Provided are an apparatus and method for testing personality based on animal symbols. The method includes outputting images of a plurality of preset animals on a screen, receiving an input for selecting a first animal group of a first number from a user, receiving an input for selecting a second animal group of a second number from the user, generating a characteristic pattern by combining characteristics of animals selected in the first animal group and the second animal group, determining a user personality type based on the generated characteristic pattern and a personality type table, and outputting the determined user personality type.

### BACKGROUND OF THE DISCLOSURE

### Field of the Disclosure

The present disclosure relates to an apparatus and method for testing personality, and more particularly, to an apparatus and method for testing personality based on animal symbols.

### Related Art

The personality test refers to a measurement method for testing personal characteristics, attitudes, or personality such as temperament, and expressing the test results in systematic numerical values. There are various types of personality tests depending on the purpose. Through the personality test, it is possible to more accurately grasp an individual, and to use the grasped information variously and usefully depending on the purpose of use. In particular, the personality test systematically determines typical personality types based on certain principles, and classifies and organizes the personality of various individuals accordingly, thereby facilitating understanding of personality.

In this way, various methods for testing personality have been presented to categorize and discern personalities of people.

Korean Patent Laid-Open Publication No. 10-2010-7013827 (published Sep. 27, 2010), “an apparatus for testing personality” discloses a technique for displaying question items of Minnesota Multiphasic Personality Inventory (MMPI) to test subjects and testing personality based on the response.

However, the above method for testing personality has the disadvantages that it is less intuitive and the questionnaire is composed of complex sentences, which reduces the concentration of the test subject and the reliability of the test results.

Therefore, in the technical field, there is a demand for a method for testing personality test capable of grasping the personality of the test subject with intuitive and easy questions.

An example of the related art includes Korean Patent Laid-Open Publication No. 10-2010-0103531, published on Sep. 27, 2010 (Title: Apparatus for Testing Personality).

### SUMMARY

The present disclosure provides an apparatus and method, or software for testing personality based on animal symbols.

The present disclosure also provides an apparatus and method, or software for testing personality, capable of grasping the personality of a test subject with intuitive and easy questions.

In an aspect, a method for testing personality based on animal symbols is provided. The method includes outputting images of a plurality of preset animals on a screen, receiving an input for selecting a first animal group of a first number from a user, receiving an input for selecting a second animal group of a second number from the user, generating a characteristic pattern by combining characteristics of animals selected in the first animal group and the second animal group, determining a user personality type based on the generated characteristic pattern and a personality type table, and outputting the determined user personality type.

In the aspect, each of the plurality of output animals may correspond to at least one of predetermined characteristics.

In the aspect, the outputting of the images of the animals may include outputting the same number of animals for each predetermined characteristic on the screen.

In the aspect, the generating of the characteristic pattern may include counting the number of times the animals are selected by the user for each group classified based on characteristics of the animals.

In the aspect, the determining of the user personality type may include determining a personality type matching the classified group that is selected most often by the user as a main personality of the user, and determining a personality type matching the classified group that is selected second most often by the user as a secondary personality of the user.

In another aspect, an apparatus for testing personality based on animal symbols is implemented. The apparatus includes an input and output unit configured to output images of a plurality of preset animals on a screen, receive an input for selecting a first animal group of a first number from a user, receive an input for selecting a second animal group of a second number from the user, and output the images of the plurality of preset animals on the screen, a processor configured to generate a characteristic pattern by combining characteristics of animals selected in the first animal group and the second animal group and determine a user personality type based on the generated characteristic pattern and a personality type table, and a memory configured to store at least one of the images of the plurality of preset animals, the first animal group and the second animal group, the generated characteristic pattern, and the personality type table.

In the aspect, each of the plurality of output animals may correspond to at least one of predetermined characteristics.

In the aspect, the input and output unit may output the same number of animals for each predetermined characteristic on the screen when the images of the animals are output.

In the aspect, the processor may count the number of times the animals are selected by the user for each group classified based on characteristics of the animals when the characteristic pattern is generated.

In the aspect, the processor may determine a personality type matching the classified group that is selected most often by the user as a main personality of the user, and may determine a personality type matching the classified group that is selected second most often by the user as a secondary personality of the user, in the determining of the user personality type.

### BRIEF DESCRIPTION OF THE DRAWINGS

FIG. 1 illustrates an example of symbolic animals to be used in the present disclosure.

FIG. 2 is a flowchart showing a method for testing personality based on animal symbols according to an embodiment of the present disclosure.

FIG. 3 illustrates an example of a question item screen for implementing a method for testing personality based on animal symbols according to an embodiment of the present disclosure.

FIG. 4 illustrates an example of a personality type result output on a screen of a user according to an embodiment of the present disclosure.

FIG. 5 is a block diagram illustrating an apparatus for testing personality based on animal symbols according to an embodiment of the present disclosure.

FIG. 6 is a block diagram illustrating an apparatus for testing personality based on animal symbols according to another embodiment of the present disclosure.

### DESCRIPTION OF EXEMPLARY EMBODIMENTS

Hereinafter, the present disclosure will be described in detail with reference to the accompanying drawings. Here, detailed descriptions of repeated descriptions, and known functions and configurations that may unnecessarily obscure the subject matter of the present disclosure are omitted. Embodiments of the present disclosure are provided to more completely describe the present disclosure to those having ordinary skill in the art. Accordingly, the shapes and sizes of elements in the drawings may be exaggerated for clearer description.

Hereinafter, preferred embodiments of the present disclosure will be described in detail with reference to the accompanying drawings.

In the present specification, a test subject undergoing a personality test according to an apparatus and method for testing personality may be defined as a ‘user’ or a ‘test subject’, and the ‘user’ or ‘test subject’ may be used interchangeably or with the same meaning.

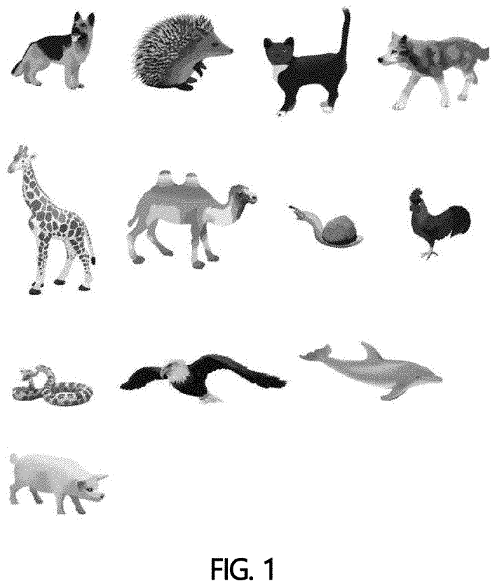

FIG. 1 illustrates an example of symbolic animals to be used in the present disclosure.

Referring to FIG. 1, each animal may have different characteristics depending on, for example, whether each animal lives in a herd, whether it lives in the wild, or whether it lives on the ground or in the air, and the different characteristics of the animals may be used to grasp the personality of the test subject. In FIG. 1, a total of 12 animals (dog, hedgehog, cat, giraffe, camel, wolf, snail, chicken, viper, eagle, dolphin, pig) are illustrated; however, those are only exemplary, and a larger number of animal images (for example, 30) may be output.

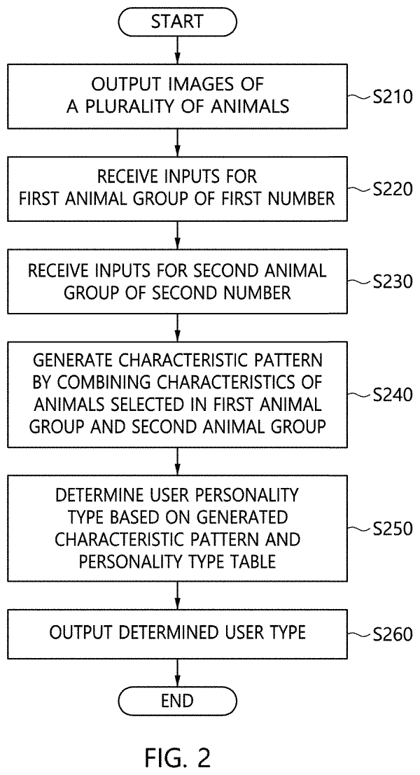

FIG. 2 is a flowchart showing a method for testing personality using an apparatus for testing personality based on animal symbols according to an embodiment of the present disclosure.

Referring to FIG. 2, the method for testing personality based on an animal symbol according to an embodiment of the present disclosure outputs images of a plurality of preset animals on a screen (S210).

In this case, on the user screen of the apparatus for testing personality, images of a plurality of animals may be output in an arrangement as illustrated in FIG. 1, for example. In FIG. 1, a total of 12 animals (dog, hedgehog, cat, giraffe, camel, wolf, snail, chicken, viper, eagle, dolphin, pig) are illustrated; however, those are only exemplary, and a larger number of animal images (for example, 30) may be output.

In this case, the plurality of output animals may each correspond to at least one of predetermined characteristics. For example, the plurality of output animals may each correspond to at least one of predetermined characteristics in Table 1 below.

For the implementation of the embodiment, a plurality of animals may be classified in advance based on their characteristics. The criteria for classifying a plurality of animals may be, for example, whether or not they live in a herd, whether or not they are wild animals, or whether or not they are ground/air animals. In addition, each of the plurality of animals corresponds to at least one of a predetermined number of personality symbolic characteristics (or personality types), as shown in Table 1 below. That is, the characteristics of each animal may correspond to the personality of the person symbolized by each animal. Table 1 below may be called a personality type table.

TABLE 1
1. Goal/Achievement: Stag, Elephant, Giraffe, Buffalo, Kangaroo
2. Ideal/Escape: Dolphin, Seal
3. Initiative/control: Polar bear, Viper, Black panther, Crocodile, Tiger
4. Application/Flexibility: Rabbit, Cow, Sheep, Horse, Camel
5. Guard/Protect: Dog, Rooster
6. Freedom/Distraction: Pig, Cat, Monkey Kitten
7. Observation/Analysis: Owl, Eagle
8. Loyalty: Wolf, Lioness
9. Alert/Defense: Hedgehog, Meercat, Snail, Fox
10. Harmony: When all types are represented in various ways

In Table 1, 30 animals correspond to, for example, 10 symbolic personalities (or personality types); however, it is merely exemplary and the number of symbolic personalities may be smaller or larger. The table in which animals are matched to symbolic characteristics, as shown in Table 1 above, may be converted into a database and stored in advance in a processor or memory.

In this case, among the plurality of output animals, the same number of animals may be output for each predetermined characteristic on the screen. For example, on the screen of the user, two animals may be selected for each type (types 1 to 9) in Table 1, and a total of 18 animals may be output on the screen.

Next, the apparatus for testing personality receives an input for selecting the first animal group of the first number from the user (S220).

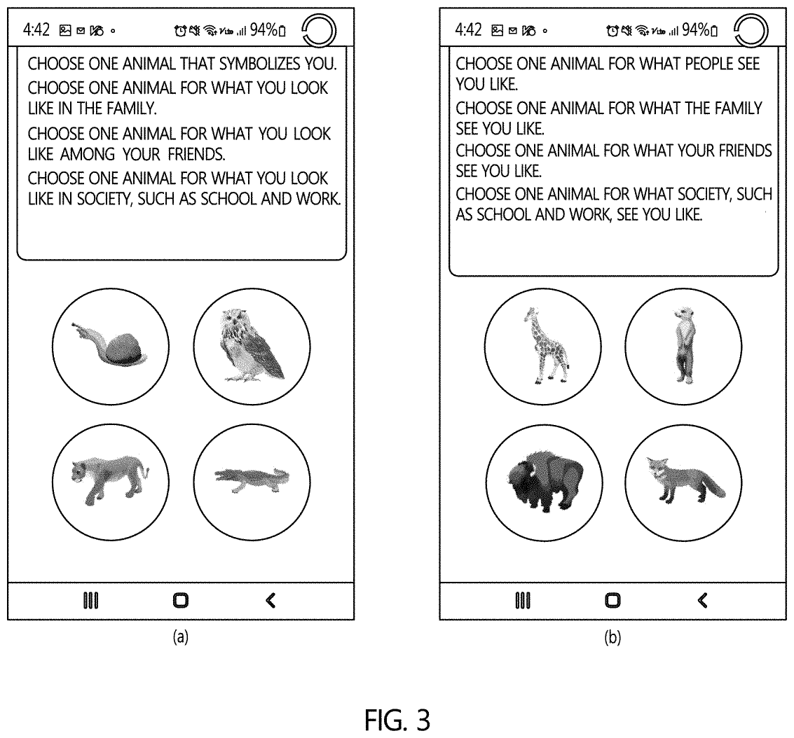

In this case, a message may be output on the screen of the user that instructs the test subject to select a first animal group reflecting the personality of the test subject from among the output animal images by a predetermined first number (for example, 4 or more). For example, on the screen of the user, questions such as “Choose one animal that symbolizes you.”, “Choose one animal for what you look like in the family.”, “Choose one animal for what you look like among your friends.”, and “Choose one animal for what you look like in society, such as school and work.” may be output. FIG. 3(a) illustrates an example in which question items for receiving inputs for selecting the first animal group of the first number in step S220 are output.

In FIG. 3(a), a case in which a plurality of questions are simultaneously displayed on the screen of the user and the selection inputs for the first animal group of the first number are received at once are illustrated; however, a case in which the selection inputs are received in a sequential manner, that is, a question is displayed on the screen of the user to select an animal and then another question is presented to select a next animal, and so on, may be provided. For example, the apparatus for testing personality presents a screen of “Choose one animal that symbolizes you” and then receives a selection input by the user. Then, the apparatus for testing personality presents the screen of “Choose one animal for what you look like in the family” on a new screen, and then receives a selection input from the user. Then, the apparatus for testing personality may output “Choose one animal for what you look like among my friends” on the next screen and then may receive a selection input, and may output “Choose one animal for what you look like in society, such as school and work” on the next screen and then may receive a selection input.

In this case, the selection input of the first number may be implemented through an input interface (e.g., a keyboard/mouse of a computer or a touch input of a display provided in a smartphone) of the apparatus for testing personality. This step may be performed by a processor of a selection input device.

Next, the apparatus for testing personality receives an input for selecting the second animal group of the second number from the user (S230).

In this case, a message may be output on the screen of the user that instructs the test subject to select the second animal group reflecting the personality of the test subject from among the output animal images by a predetermined second number (for example, 4 or more). For example, on the screen of the user, questions such as “Choose one animal for what people see you like.”, “Choose one animal for what the family see you like.”, “Choose one animal for what your friends see you like.”, and “Choose one animal for what society, such as school and work, see you like” may be output. FIG. 3(b) illustrates an example in which question items for receiving inputs for selecting the second animal group of the second number in step S230 are output.

In FIG. 3(b), a case in which a plurality of questions are simultaneously displayed on the screen of the user and the selection inputs for the second animal group of the second number are received at once are illustrated; however, a case in which the selection inputs are received in a sequential manner, that is, a question is displayed on the screen of the user to select an animal and then another question is presented to select the next animal, and so on, may be provided. For example, the apparatus for testing personality presents a screen of “Choose one animal for what people see you like” and then receives a selection input by the user. Then, the apparatus for testing personality presents the screen of “Choose one animal for what the family see you like.” on a new screen, and then receives a selection input from the user. Then, the apparatus for testing personality may output “Choose one animal for what your friends see you like.” on the next screen and then may receive a selection input, and may output “Choose one animal for what society, such as school and work, see you like.” on the next screen and then may receive a selection input.

In this case, the selection input of the second number may be implemented through an input interface (e.g., a keyboard/mouse of a computer or a touch input of a display provided in a smartphone) of the apparatus for testing personality. This step may be performed by a processor of a selection input device.

Next, the apparatus for testing personality generates a characteristic pattern by combining characteristics of animals selected in the first animal group and the second animal group (S240).

The apparatus for testing personality generates a characteristic pattern based on Table 1 and information on the first animal group and the second animal group selected by the test subject. The generating of the characteristic pattern according to the example may include counting the number of the characteristics that have been derived by repetition. For example, the counting is performed such as the number of characteristics that are found most often is 5, and the number of characteristics that are found second most often is 3. An example of characteristic patterns (or test result patterns) derived by the method is shown in Table 2 below.

TABLE 2
7.1
6.2
6.1.1
5.1.1.1
5.2.1
5.3
4.1.1.1.1
4.3.1
4.2.1.1
4.2.2
3.2.2.1
3.2.1.1.1
3.3.1.1 (in this case, there are two main personalities)
3.1.1.1.1.1
2.2.1.1.1.1
2.2.2.2
2.1.1.1.1.1.1
2.2.2.1.1
1.1.1.1.1.1.1.1

Next, the apparatus for testing personality determines a user personality type based on the generated characteristic pattern and a personality type table (S250).

When interpreting Table 2 with reference to Table 1, the underlined numbers in Table 2 represents all types in various ways, and thus correspond to the number 10 (harmony) type in Table 1. That is, when the number of types selected most often by the user in each generated pattern is two or less, or the number of types selected most often is four or less and the number of each of other types selected is only one without overlapping each other, it may be determined that the user personality type corresponds to the number 10 (harmony) type.

In addition, in Table 2, dark numbers are the most frequent type. The number 1 may omit its meaning. That is, the user personality type may be effectively treated only when it matches the characteristic pattern that has received two or more selections from the user.

In addition, personality types matching the animals selected in step S220 may be determined as subjective personality types, and personality types matching the animals selected in step S230 may be determined as objective personality types.

Next, the apparatus for testing personality outputs the personality type determined in step S250 on the screen (S260).

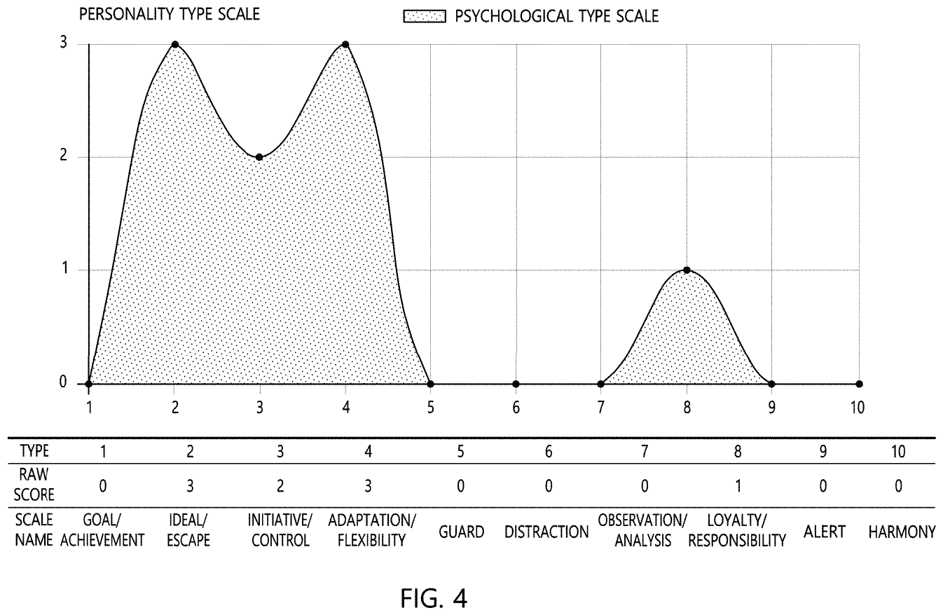

FIG. 4 illustrates an example of a personality type result output on a screen of a user according to an embodiment of the present disclosure. Referring to FIG. 4, independently of the personality type of the user being output, each psychological characteristic scale may be output as a statistical graph based on animals selected by the user on the screen of the user.

Meanwhile, the results shown in the example of FIG. 4 indicate a case where three animals are selected for each of personalities representing the types of reality/escape (Number 2) and application/flexibility (Number 4). In this case, reality/escape (Number 2) and application/flexibility (Number 4) may be the main personality of the user, and accordingly, two personalities may be determined and output as the main personality.

Meanwhile, the personality type output on the screen may include at least one of a main personality type and an auxiliary personality type. For example, in Table 2, the personality type in which the animal characteristic is found most often may be determined as the main personality type, and the personality type in which the animal characteristic is found second most often may be determined as the auxiliary personality type.

In this case, as shown in 3.3.1.1 in Table 2, when there are two of the most frequent frequencies, it may be determined that the main character is two in number.

Table 3 below is an example of the test results by the apparatus for testing personality.

TABLE 3

| Type | Test Results |
|---|---|
| Goal/ | Being personality for running for goals and |
| Achievement | achievements. Running toward a certain goal, and |
| (No. 1) Type | being productive and trying to achieve something. |
| Ideal/Escape | Lacking a sense of reality, having an escaped |
| (No. 2) Type | attitude, and having an idealistic attitude. |
| Initiative/Control | Pursuing independence, freedom, and initiative, and |
| (No. 3) Type | seeking to have power, dominance, and control. |
| Application/ | Being excellent adaptability and flexibility. Being |
| Flexibility | gentle, compliant, and passive. |
| (No. 4) Type | |
| Guard/Protect | Basically, being conservative and trying to uphold |
| (No. 5) Type | principles, values, and cultures considered to be |
| | important. |
| Freedom/ | Pursuing independence, freedom, and initiative, |
| Distraction | and feeling a sense of resistance to being bound. |
| (No. 6) Type | |
| Observation/ | Always trying to be objective and neutral. |
| Analysis | |
| (No. 7) Type | |
| Loyalty (No. 8) | Being high loyal and responsible. |
| Type | |
| Alert/Defense | Being very alert. Alert and defense being the most |
| (No. 9) Type | important parts of life. |
| Harmony | Developing several personality types in order to |
| (No. 10) Type | avoid tensions and conflicts and live in harmony. |

The apparatus for testing personality may display analysis results for the personality test through the display screen. The following is an example of the analysis results for the personality test provided to the test subject. The display screen may be displayed with being divided into main personality and secondary personality.

Main Personality

1. Goal/Achievement Type

The test subject is extroverted, and considers goals and achievements most important. The test subject runs toward a certain goal, is productive, and tries to achieve something. The test subject pursues independence, freedom and initiative, and is docile, relationship-oriented, and sociable. The test subject values a relationship rather than trying to control or dominate. The test subject values the affiliated group and is willing to take on the role in the group. The test subject is confident in his or her optimism and is active, enthusiastic, and efficient in everything. The test subject is passionate and practical. On the other hand, the test subject is competitive and pretended, paying too much attention to the gaze of others. The test subject is stubborn and difficult to bear with other people's mistakes.

The strength is that the test subject is quick to grasp information, efficient and skillful. The test subject is optimistic, friendly, and cheerful, and has good relationships with those around them. The test subject motivates people well, overcomes failures quickly and moves on to the next task. The weakness is that the test subject is obsessed with success and cannot stand ineffective or incompetent. The test subject is exhausted because he or she always run toward new goals and achievements. The test subject is impatient and impetuous.

2. Ideal/Escape Type

The test subject is extroverted, seeks independence, freedom, and initiative, tries to find meaning in life, and acts with inner motives to get out of everyday life. The test subject has an idealistic attitude, lacks a sense of reality, and has a tendency to escape reality. The test subject is able to reflect on himself or herself, and is intuitive and creative. The test subject has a variety of curiosities and is relationship-oriented. On the other hand, the test subject tends to be immersed in himself or herself and feels depressed, helpless and guilty. Also, the test subject has severe emotional ups and downs.

The strength is that the test subject has high creativity and intuition and something unique, so looks unique to other people. The test subject is considered as a funny and warm person by being cheerful and expressing his or her feelings well. The test subject pursues the meaning and value of life and has a noble attitude about life. On the other hand, the weakness is that the test subject has so severe emotional ups and downs that the test subject easily experiences depressed feelings such as emptiness or despair. Due to a lack of reality, the test subject craves what he or she does not have and suffer from high expectations of himself or herself.

3. Initiative/Control Type

The test subject is introverted, seeks independence, freedom, and initiative, and tries to gain power, dominance and control. The test subject does not value relationships, and is only goal-oriented and fulfilling. The test subject always has a goal of having power, dominance, and control, and considers the needs for survival and safety important. Basically, the test subject has high anxiety and cannot get along well with others. Instead, the test subject wants to take initiative and power. The test subject wants to be independent and strong and has an inner motive to avoid feelings of being dependent or weak.

The test subject is well established in Independence and separation above all. The test subject is realistic, enthusiastic, and energetic. On the other hand, the test subject uses direct communication and is aggressive and critical. The test subject has a selfish, rebellious, and uncompromising tendency.

The strength is that the test subject is brave and challenging, and runs toward a certain goal. The test subject is honest and initiatively faces a problem without avoiding it. The weakness is that the test subject tries to take the initiative in every situation. When the test subject does not follow his or her own rules and frameworks, he or she is very hard. The test subject is picky, arrogant, combative, and possessive. The test subject exerts excessive pressure on himself or herself and others. The test subject hates to suffer damages or be treated unfairly. When angry, the test subject hurts others because he or she attacks the other person directly.

4. Adaptation/Passive Type

The test subject is extroverted, and has excellent adaptability and flexibility. The test subject is gentle, compliant, and passive. The test subject lack self-direction and is unable to make self-assertion. Instead, the test subject follows the needs of others and the given framework. The test subject has a dependent characteristic, is relationship-oriented, and is faithful to his or her responsibilities and duties. The test subject willingly takes care of and sacrifices those around himself or herself. Basically, the test subject pursues law and order, hierarchical order, harmony, harmony, stability, and a sense of belonging. Traditionally, women rather than men were required to have this trait. The test subject is friendly and takes good care of people. The test subject has insight and are sensitive to the emotions of people. The test subject follows the framework and is compliant. On the other hand, the test subject uses indirect communication and is overly compliant and passive. The test subject tries to make an adjustment from behind and expresses his or her emotions violently or are nervous.

The strength is that the test subject forms relationships well and have insight such that he or she adapts well to their surroundings and learning or learning processes, and flexibly copes with the given framework. The test subject is aware of the feelings of others, is generous, takes good care of others, and has a warm heart. The test subject is aware of the needs of people and willingly helps them. The test subject has the characteristics of a model student. The weakness is that the test subject is dependent and difficult to be independent and separate. The test subject studies other's face, is unable to easily refuse, and blames himself or herself. The test subject has a strong desire to possess, but he or she is afraid of becoming selfish, and thus he is not able do what he wants to do. The test subject really suppresses his or her feelings because he or she spends all energy adjusting to the surroundings. The test subject is tired of striving for other people.

5. Guard/Protect Type

The test subject is both extroverted and introverted, are conservative, and try to uphold principles and values and cultures that are considered important. The test subject has a personality that has both aggression, domination, control, strength, and active attitudes, as well as adaptive, gentle, and passive attitudes. The test subject values the sense of belonging within the group, adheres to the principles, and tries to live his or her life properly. The test subject is ethical, smart and fair. The test subject is honest and self-disciplined well, so he or she has a dependable personality. On the other hand, the test subject tries to judge, is assertive, and is inflexible. The test subject is to be critical or overly serious and tries to control. The test subject has high anxiety and suffers from obsessive thoughts and impulses. The strength is that the test subject has high standards and ethical consciousness, and is reasonable, responsible, and dedicated. The test subject is faithful, dedicated, conscientious, and helpful. The test subject has appropriate self-discipline and hard-working attitudes and is good at accomplishing his or her given task. The test subject thinks integrative, is understandable, and presents wise solutions. The weakness is that the test subject suffers burdened and obsessive due to excessive responsibility. When the test subject fails to meet expectations due to his or her perfectionist tendencies, the test subject is disappointed in himself or herself and others. The test subject is nervous, worried, and overly serious in everything. The test subject is critical, tries to argue, is determined, and gets caught up in all cases. The test subject demands high standards from himself or herself and others.

6. Freedom/Distraction Type

The test subject is both extroverted and introverted. The test subject considers individual freedom more important than responsibility and belonging, and prioritizes personal characteristics over relationships. The test subject feels a strong resistance to being bound. The test subject refuses to be controlled and dominated, and hates to carry out routine tasks within a group. The test subject plans interesting things, tries to be happy, avoids pain and agony, and has distraction and impulsive characteristics. The test subject seeks pleasure, is curious, is spontaneous, is creative, and is imaginative. On the other hand, the test subject is impulsive, distracted, and arbitrary. The test subject is rebellious, overly excited, and self-destructive. The test subject has a narcissistic tendency and a strong desire to possess.

The strength is that the test subject is curious, versatile, and interested. The test subject is optimistic, is spontaneous, has a free spirit, talks without hesitation, and behaves weirdly. The test subject is active, is adventurous, livens up the mood, and entertains people. The weakness is that the test subject is distracted, impulsive, narcissistic, and arbitrary. The test subject does not complete the planned work and is unrealistic. The test subject insists on own opinions and is defensive. The test subject hates being bound by the other person in a relationship.

7. Observation/Analysis Type

The test subject has an introverted tendency. The test subject always observes and analyzes from a distance, rather than having a direct relationship within a group. The test subject tries to grasp everything and not to look silly. The test subject is cautious in everything and does not try to reveal himself. The test subject takes a step back and collects information. The test subject is objective, neutral, wise, and insightful. The test subject has a special relationship with a small number of friends, rather than making many friends. The test subject is smart and curious. The test subject solves problems wisely by himself or herself, and is prudent and patient. On the other hand, the test subject is stingy, stubborn, and proud. The test subject does not insist strongly, and does not relax alert to avoid getting caught up in the opponent. The test subject does not trust the other person well, and is critical and negative.

The strength is that the test subject grasps the causal relationship of work and thoroughly understand it, and responds calmly to crisis situations. The test subject does not cling to material possessions or status, and has a truthfulness in doing what he or she thinks is right without being subject to pressure. The test subject has an eye for recognizing knowledge and skills.

The weakness is that the test subject has difficulty revealing his or her knowledge and insights. It is difficult to communicate deeply with others because the test subject avoids revealing his or her feelings. The test subject feels that his or her territory is invaded and controlled, and that the other person ignores the test subject. When the test subject does something he or she does not want, the test subject gets a lot of stress. The test subject is sensitive and wants to avoid conflicts that arise in human relationships. To avoid being noticed that the test subject is afraid, the test subject has an expressionless face.

8. Loyalty Type

The test subject is extroverted, faithful and responsible. The test subject considers a sense of responsibility and belonging important above all. The test subject values the affiliated group and is willing to take on the role in the group. The test subject values the group rather than his or her own individual part, and tries to faithfully follow the values and duties of the group. The needs for survival and safety act as an inner motive, and the needs for survival and safety make the test subject conform to the group and willingly take on responsibility. The test subject is loyal, practical, and prudent. The test subject is witty, warm, and helpful. The test subject confronts danger bravely. The test subject respects the authorities who can protect himself or herself. On the other hand, the test subject strictly distinguishes between people in his or her own group or group and those who are not. The test subject decides too carefully, is defensive, and tries to control. The test subject is cynical, arrogant, and stubborn, and is not conforming. On the contrary, if the test subject does not respect the authority, the test subject is rebellious and does not try to acknowledge authorities.

The strength is that the test subject is highly responsible, faithful and dedicated in family, friends and work. The test subject has a high sense of community and forms a group well. The weakness is that the test subject is defensive and exclusive to those outside the group and the environment, and tries to control and decide those within the group. The test subject is afraid of being abandoned or used by others. If the test subject does things that do not meet the expectations of others, the test subject violently rebukes himself or herself. The test subject is inflexible, delusional, and exacerbating the problem. The test subject is very irritable.

9. Alert/Defense Type

The test subject is introverted and considers vigilant the most important. The test subject is defensive and tries to keep his or her strength and initiative to protect himself or herself. The need for survival and safety act as an inner motive. Basically, the test subject has high anxiety, does not trust others, and does not get along well with others. The test subject is wary so that others do not dominate and control himself or herself. The test subject has the typical personality type that people who experience unstable attachment have. The test subject has all nerves to be wary of others, and is isolated or play alone. The biggest reason to be wary tries not to get under the control of others. The test subject does not have a wide range of interpersonal relationships. The test subject is very careful when he or she contacts only a few close people and makes friends. The test subject has a defensive attitude toward life and adheres to his or her own way of life and human relationships.

The strength is that the test subject is independent, self-reliant, careful and prudent. The test subject has a certain pattern of his or her own behavior. The weakness is that the test subject avoids tensions and conflicts while studying other's face. The test subject tends to be overly suspicious of others. The test subject is too defensive and very aggressive when he or she feels that his or her boundaries have been violated or his or her territory has been violated. When the test subject does not follow his or her own rules and frameworks, he or she is very hard. The test subject never forgets to suffer damages or be treated unfairly.

10. Harmony Type

Extroversion and introversion come together. The test subject wants to live in harmony, avoids tensions and conflicts, and acts according to his or her desire to get along with people. The test subject is unable to endure tensions and conflicts and tries to avoid them. The test subject is a person who has not developed his or her own unique personality in order to adapt to the difficult environment in his childhood, but developed various personalities for adaptation. Since there are multiple personality types within a person, it is difficult for the test subject to define his or her personality clearly. The test subject has the inner motive of wanting to get along with people and has a variety of personality characteristics ranging from mild to strong and independent personality types.

The advantage is that the test subject is seen as a good person, and accepts something as it is without determining. The test subject takes care of people and pays attention to them. The test subject does not show negative emotions outwardly. The test subject makes people enjoyable and comfortable. The test subject has the function of a mediator because he or she sees the problem from various perspectives. The test subject has a sensuous and aesthetic perspective and has an excellent sense of reality. The weakness is that the test subject may be overly quiet and indecisive. The test subject lacks determination and self-control and distraction. The test subject is a little excited and may be confusing. The test subject has low self-esteem, is very sensitive to criticism, and is easily hurt. The test subject is confused about what he or she wants. The test subject pays too much attention to people's gaze and evaluation. The test subject tries to avoid or turn off his or her nerves when tensions and conflicts arise.

Secondary Personality

1. Goal/Achievement Type

The test subject is extroverted, and considers goals and achievements most important. The test subject runs toward a certain goal, and is productive and tries to achieve something. The test subject pursues independence, freedom and initiative, and is docile, relationship-oriented, and sociable. The test subject values a relationship rather than trying to control or dominate. The test subject values the affiliated group and is willing to take on the role in the group. The test subject is confident in his or her optimism and is active, enthusiastic, and efficient in everything. The test subject is passionate and practical. On the other hand, the test subject is competitive and pretended, paying too much attention to the gaze of others. The test subject is stubborn and difficult to bear with other people's mistakes.

2. Ideal/Escape Type

The test subject is extroverted, seeks independence, freedom, and initiative, tries to find meaning in life, and acts with inner motives to get out of everyday life. The test subject has an idealistic attitude, lacks a sense of reality, and has a tendency to escape reality. The test subject is able to reflect on himself or herself, and is intuitive and creative. The test subject has a variety of curiosities, is relationship-oriented, and is warm and supportive in relationships. On the other hand, the test subject tends to be immersed in himself or herself and feels depressed, helpless and guilty. Also, the test subject has severe emotional ups and downs.

3. Initiative/Control Type

The test subject is introverted, seeks independence, freedom, and initiative, and tries to gain power, dominance and control. The test subject does not value relationships, and is only goal-oriented and fulfilling. The test subject always has a goal of having power, dominance, and control, and considers the needs for survival and safety important. Basically, the test subject has high anxiety and cannot get along well with others. Instead, the test subject wants to take initiative and power. The test subject wants to be independent and strong and has an inner motive to avoid feelings of being dependent or weak. The test subject is well established in Independence and separation above all. The test subject is realistic, enthusiastic, and energetic. On the other hand, the test subject uses direct communication and is aggressive and critical. The test subject has a selfish, rebellious, and uncompromising tendency.

4. Adaptation/Passive Type

The test subject is extroverted, and has excellent adaptability and flexibility. The test subject is gentle, compliant, and passive. The test subject lack self-direction and is unable to make self-assertion. Instead, the test subject follows the needs of others and the given framework. The test subject has a dependent characteristic, is relationship-oriented, and is faithful to his or her responsibilities and duties. The test subject willingly takes care of and sacrifices those around himself or herself. Basically, the test subject pursues law and order, hierarchical order, harmony, harmony, stability, and a sense of belonging. Traditionally, women rather than men were required to have this trait. The test subject is friendly and takes good care of people. The test subject has insight and are sensitive to the emotions of people. The test subject follows the framework and is compliant. On the other hand, the test subject uses indirect communication and is overly compliant and manipulated. The test subject expresses his or her emotions violently or is nervous.

5. Guard/Protect Type

The test subject is both extroverted and introverted, are conservative, and try to uphold principles and values and cultures that are considered important. The test subject has a personality that has both aggression, domination, control, strength, and active attitudes, as well as adaptive, gentle, and passive attitudes. The test subject values the sense of belonging within the group, adheres to the principles, and tries to live his or her life properly. The test subject is ethical, smart and fair. The test subject is honest and self-disciplined well, so he or she has a dependable personality. On the other hand, the test subject tries to judge, is assertive, and is inflexible. The test subject is to be critical or overly serious and tries to control. The test subject has high anxiety and suffers from obsessive thoughts and impulses.

6. Freedom/Distraction Type

The test subject is both extroverted and introverted. The test subject considers individual freedom more important than responsibility and belonging, and prioritizes personal characteristics over relationships. The test subject feels a strong resistance to being bound. The test subject refuses to be controlled and dominated, and hates to carry out a certain role given to the test subject within a group. The test subject plans interesting things, tries to be happy, avoids pain and agony, and has distraction and impulsive characteristics. The test subject is curious, is spontaneous, is creative, and is imaginative. The test subject seeks fun, strives for fun, and is agile. On the other hand, the test subject is impulsive, distracted, and arbitrary. The test subject is rebellious, overly excited, and self-destructive. The test subject has a narcissistic tendency and a strong desire to possess.

7. Observation/Analysis Type

The test subject has an introverted tendency. The test subject always observes and analyzes from a distance, rather than having a direct relationship within a group. The test subject tries to grasp everything and not to look silly. The test subject is cautious in everything and does not try to reveal himself or herself. The test subject takes a step back and collects information. The test subject is objective, neutral, wise, and insightful. The test subject has a special relationship with a small number of friends, rather than making many friends. The test subject is smart and curious. The test subject solves problems wisely by himself or herself, and is prudent and patient. On the other hand, the test subject is stingy, stubborn, and proud. The test subject does not insist strongly and does not relax alert to avoid getting caught up in the opponent. The test subject does not trust the other person well, and is critical and negative.

8. Loyalty Type

The test subject is extroverted, faithful, and responsible. The test subject considers a sense of responsibility and belonging important above all. The test subject values the affiliated group and is willing to take on the role in the group. The test subject values the group rather than his or her own individual part, and tries to faithfully follow the values and duties of the group. The needs for survival and safety act as an inner motive, and the needs for survival and safety make the test subject conform to the group and willingly take on responsibility. The test subject is loyal, practical, and prudent. The test subject is witty, warm, and helpful. The test subject confronts danger bravely. The test subject respects the authorities who can protect himself or herself. On the other hand, the test subject strictly distinguishes between people in his or her own group or group and those who are not. The test subject decides too carefully, is defensive and tries to control. The test subject is cynical, arrogant, and stubborn, and is not conforming. The test subject rebels without acknowledging authorities.

9. Alert/Defense Type

The test subject is introverted and considers vigilant the most important. The test subject is defensive and tries to keep his or her strength and initiative to protect himself or herself. The need for survival and safety act as an inner motive. Basically, the test subject has high anxiety, does not trust others, and does not get along well with others. The test subject is wary so that others do not dominate and control himself or herself. The test subject has the typical personality type that people who experience unstable attachment have. The test subject has all nerves to be wary of others, and is isolated or play alone. The biggest reason to be wary tries not to get under the control of others. The test subject does not have a wide range of interpersonal relationships. The test subject is very careful when he or she contacts only a few close people and makes friends. The test subject has a defensive attitude toward life and adheres to his or her own way of life and human relationships.

10. Harmony Type

Extroversion and introversion come together. The test subject wants to live in harmony, avoids tensions and conflicts, and acts according to his or her desire to get along with people. The test subject is unable to endure tensions and conflicts and tries to avoid them. The test subject is a person who has not developed his or her own unique personality in order to adapt to the difficult environment in his childhood, but developed various personalities for adaptation. Since there are multiple personality types within a person, it is difficult for the test subject to define his or her own personality clearly. The test subject has the inner motive of wanting to get along with people and has a variety of personality characteristics ranging from mild to strong and independent personality types.

The apparatus for testing personality according to the present disclosure may display a suggestion for each personality on the display screen. The following are examples of suggestions for each personality.

Suggestions for Each Personality

1. Goal/Achievement Type

The test subject needs to have a time of enjoying satisfying himself or herself with the success he or she has achieved. It is because the energy of his or her is not infinite. It is necessary to explore his or her own needs rather than being too preoccupied with what other people expect and demand of the test subject.

2. Ideal/Escape Type

The test subject needs to work hard to stay in reality, learns to trust himself or herself, and tries to restore his or her self-esteem. In addition, it is necessary to promote to show creativity, intuition, cheerfulness, and funniness, which are the positive aspects of personality types.

3. Initiative/Control Type

The test subject can become a ruler who has won power and leadership, or the test subject can become a lonely man who has been defeated and pushed out of everything. In the struggle for leadership and competition for power, it is necessary to empathize with the emotions and desires of the other person, rather than focusing too much on the test subject's own emotions and needs. It is important to know that the real power lies in the network and comes from smooth and in-depth communication with others.

4. Adaptation/Passive Type

Behind the ability to empathize with others, the test subject needs to know that he or she has suppressed and sacrificed his or her feelings. What is needed here is a psychological balance. The test subject needs to be aware that being generous with others and taking good care of others is not something he or she can achieve at his or her own expense. The ability to respect others needs to be extended to the ability to respect himself or herself.

5. Guard/Protect Type

In order to establish a stable relationship with others, the wisdom of integration is necessary.

In the relationship, it is necessary to try to see the whole aspect, not to evaluate and judge only one part of the other person. The test subject is to look at the other side, not decide the whole by just looking at the part disappointing himself or herself, when the test subject feels disappointed or frustrated. This integrated attitude is a source of energy that leads to good understanding and wise solutions. This will further strengthen the attitude to help people and dedicate yourself by self-discipline and hard work.

6. Freedom/Distraction Type

An unrealistic, stubborn, defensive attitude may promote the positive aspects of a personality through an effort to strengthen the adaptation and flexibility needed in a relationship. It is good to belong to a group or field that requires a highly curious, spontaneous, creative, and imaginative personality.

7. Observation/Analysis Type

There is a task to recognize and express real feelings and thoughts. The test subject may achieve an inner balance when he or she feels the intimacy and belonging that he or she may get in a relationship by adjusting the desire to go back and observe in the relationship.

8. Loyalty Type

It is necessary to strengthen the function of pliability and flexibility. Efforts are needed to relax the strict standards for outsiders and insiders and to be open.

9. Alert/Defense Type

It is necessary to realize that the source of loneliness and alienation lies in the test subject, not others. It is necessary to recognize that other people are not something to be careful and wary of all the time, but something that is able to give the test subject a sense of intimacy, solidarity, and belonging.

10. Harmony Type

The test subject has developed various personality types to adapt to various environments and people due to his or her inner motive to get along with people, but he or she has been unable to develop his or her own unique personality. The test subject is to be able to direct energy that is only directed at others to himself or herself. The test subject needs opportunities for growth to respect our feelings and thoughts and to value ourselves.

The apparatus for testing personality according to the present disclosure may display a recommended job based on each personality on the display screen. The following shows examples of recommended jobs according to each personality.

Recommended Job

1. Goal/Achievement Type

Businessman, CEO, professor, teacher, doctor, broadcaster or actor, Legal, financial, or political worker, program developer, scientist, researcher, and public official

2. Ideal/Escape Type

Inventor who is able to show creativity, artist such as music, art, dance, acting, performance planner, broadcast culture worker, literary writer, poet, educator who teaches arts and literature, social worker, psychologist, counselor

3. Initiative/Control Type

Businessman, CEO, group representative, military personnel, legal professional, union leader, politician, sports worker, teacher, medical worker

4. Adaptation/Passive Type

Social worker, civil servant, medical worker, teacher, counselor, religious person, secretary, consultant

5. Guard/Protect Type

Public servant, police officer, legal professional, education, health or medical worker, professional (accountant, or the like), banker, stock broker

6. Freedom/Distraction Type

Travel reporter, travel agent, job with many domestic or overseas business trips, freelancer, people with their own occupational areas and spaces such as a work room, consultant, conflict and dispute mediator, entertainer, non-repetitive job, job that challenges something and deal with emergencies, emergency room medical personnel, firefighter

7. Observation/Analysis Type

Experts in science and technology specialties, occupations related to crisis response, areas where analytical skills are required to solve problems, commentator, counselor, or profiler

8. Loyalty Type

This type of personality may be found in all professions. It may be found in various places such as legal, military, business, and academic.

9. Alert/Defense Type

Literary writer, civil servant, free job, professor

10. Harmony Type

Conflict mediator, diplomat, bureaucratic organization, public official, academic, patent attorney

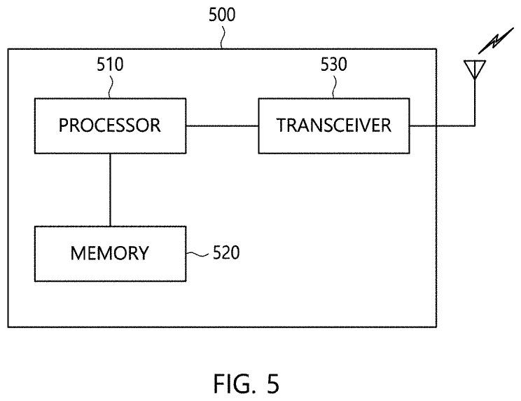

FIG. 5 is a block diagram illustrating an apparatus for testing personality based on animal symbols according to an embodiment of the present disclosure.

Referring to FIG. 5, the apparatus for testing personality according to the present embodiment includes a processor 510, a memory 520, and a transceiver 530.

The processor 510 may include an application-specific integrated circuit (ASIC), another chipset, a logic circuit, and/or a data processing device. The processor 510 implements the method for testing personality presented in the present specification. The processor 510 specifically implements all the operations of the method for testing personality described in the embodiments disclosed herein, and performs all the operations of the method for testing personality according to FIG. 2.

For example, the processor 510 outputs images of a plurality of preset animals on a screen, receives an input for selecting a first animal group of a first number from the user, receives an input for selecting a second animal group of a second number from the user, generates a characteristic pattern by combining characteristics of animals selected in the first animal group and the second animal group, determines a user personality type based on the generated characteristic pattern and a personality type table, and outputs the determined user personality type.

In this case, the plurality of output animals may each correspond to at least one of predetermined characteristics.

In this case, the processor 510 may output the same number of animals for each predetermined characteristic on the screen when the images of the animals are output.

In this case, the processor 510 may count the number of times the animals are selected by the user for each group classified based on characteristics of the animals when the characteristic pattern is generated.

In this case, the processor 510 may determine a personality type matching the classified group that is selected most often by the user as a main personality of the user, and may determine a personality type matching the classified group that is selected second most often by the user as a secondary personality of the user, in the determining of the user personality type.

In this case, the processor 510 may effectively treat the user personality type only when two or more characteristic types are selected by the user, in the determining of the user personality type.

In this case, the processor 510 may determine the user personality type as No. 10 (harmony type) when the number of types selected most often by the user in each generated characteristic pattern is two or less, or the number of types selected most often by the user is four or less and the number of each of other types selected is only one without overlapping each other, in the determining of the user personality type.

The transceiver 530 is connected to the processor 510 and transmits and/or receives information necessary for the method for testing personality by the apparatus for testing personality. For example, the transceiver 530 may receive, from an external device or an external server, at least one of images of a plurality of preset animals and the personality type table, the main personality and secondary personality based on the user personality, suggestions, and recommended jobs.

The memory 520 may include a read-only memory (ROM), a random access memory (RAM), a flash memory, a memory card, a storage medium, and/or other storage devices. In this case, the memory 520 stores at least one of images of a plurality of preset animals, the first and second animal groups selected by the user, the personality type table, the main personality and the secondary personality based on the user personality type, suggestions, and recommended jobs.

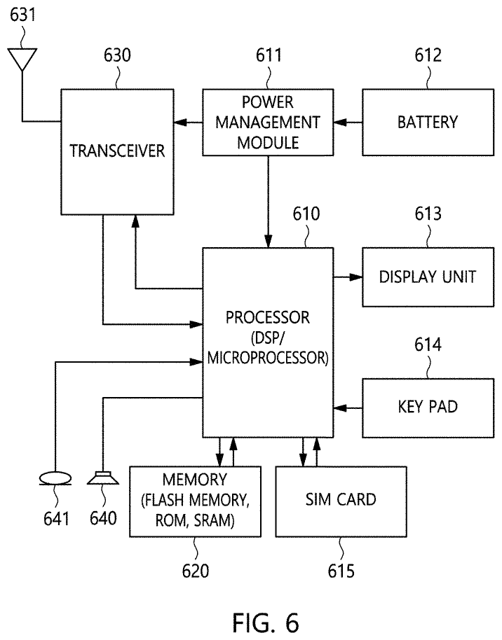

FIG. 6 is a block diagram illustrating an apparatus for testing personality based on animal symbols according to another embodiment of the present disclosure.

A user equipment (UE) is a processor 610, a power management module 611, a battery 612, a display 613, a keypad 614, a subscriber identification module (SIM) card 615, a memory 620, a transceiver 630, one or more antennas 631, a speaker 640, and a microphone 641.

The processor 610 may be configured to implement the presented functions, procedures and/or methods described herein. The layers of a radio interface protocol may be implemented by the processor 610. The processor 610 may include an application-specific integrated circuit (ASIC), another chipset, a logic circuit, and/or a data processing device. The processor may be an application processor (AP). The processor 610 may include at least one of a digital signal processor (DSP), a central processing unit (CPU), a graphics processing unit (GPU), and a modem (modulator and demodulator). Examples of the processor 610 include SNAPDRAGON™ series processors manufactured by Qualcomm®, EXYNOS™ series processors manufactured by Samsung®, A series processors manufactured by Apple®, HELIO™ series processors manufactured by MediaTek®, ATOM™ series processors manufactured by INTEL® or corresponding next-generation processors.

The processor 510 may be configured to output images of a plurality of preset animals on a screen, receives an input for selecting a first animal group of a first number from the user, receive an input for selecting a second animal group of a second number from the user, generate a characteristic pattern by combining characteristics of animals selected in the first animal group and the second animal group, determine a user personality type based on the generated characteristic pattern and a personality type table, and output the determined user personality type.

In this case, the plurality of output animals may each correspond to at least one of predetermined characteristics.

In this case, the processor 610 may output the same number of animals for each predetermined characteristic on the screen when the images of the animals are output.

In this case, the processor 610 may count the number of times the animals are selected by the user for each group classified based on characteristics of the animals when the characteristic pattern is generated.

In this case, the processor 610 may determine a personality type matching the classified group that is selected most often by the user as a main personality of the user, and may determine a personality type matching the classified group that is selected second most often by the user as a secondary personality of the user, in the determining of the user personality type.

In this case, the processor 610 may effectively treat the user personality type only when two or more characteristic types are selected by the user, in the determining of the user personality type.

In this case, the processor 610 may determine the user personality type as No. 10 (harmony type) when the number of types selected most often by the user in each generated characteristic pattern is two or less, or the number of types selected most often by the user is four or less and the number of each of other types selected is only one without overlapping each other, in the determining of the user personality type.

The power management module 611 manages power for the processor 610 and/or the transceiver 630. The battery 612 supplies power to the power management module 611. The display 613 outputs the result processed by the processor 610. The keypad 614 receives inputs to be used by processor 610. The keypad 614 may be displayed on the display 613. The SIM card 615 is an integrated circuit used to securely store an international mobile subscriber identity (IMSI) used to identify and authenticate a subscriber in a mobile phone device such as a mobile phone and a computer and a key associated therewith. A lot of contact information may be stored on the SIM card.

The memory 620 is operatively coupled with the processor 610 and stores various information for operating the processor 610. The memory 620 may include a read-only memory (ROM), a random access memory (RAM), a flash memory, a memory card, a storage medium, and/or other storage devices. When the embodiments are implemented as software, the techniques described in the present specification may be implemented as a module (e.g., a procedure, a function, and the like) that performs the functions described in the present specification. Modules may be stored in the memory 620 and executed by the processor 610. The memory 620 may be implemented inside the processor 610. Alternatively, the memory 620 may be implemented outside the processor 610 and may be communicatively connected to the processor 610 through various means known in the art.

The transceiver 630 is operatively coupled to the processor 610, and transmits and/or receives a radio signal. The transceiver 630 includes a transmitter and a receiver. The transceiver 630 may include a baseband circuit for processing radio frequency signals. The transceiver controls one or more antennas 631 to transmit and/or receive radio signals.

The speaker 640 outputs a sound-related result processed by the processor 610. The microphone 641 receives a sound-related input to be used by the processor 610.

As described above, an apparatus and method for testing personality based on animal symbols according to the present disclosure are not limited to the configurations and methods of the above-described embodiments, and various modifications to the embodiments may be made by selectively combining all or some of the embodiments.

According to the present disclosure, an apparatus and method, or software for testing personality based on animal symbols are provided.

In addition, the personality of a test subject may be grasped with intuitive and easy questions.

What is claimed is:

1. A method for testing personality based on animal symbols by an apparatus for testing personality, the method comprising:
outputting images of a plurality of preset animals on a screen;
outputting a first question for inducing a user to select an animal representing the user, from among the images of the plurality of preset animals;
receiving an input for selecting a first animal group of a first number from the user in response to the first question, the first animal group being used as subjective determination data for determining a personality type of the user;
re-outputting the images of the plurality of preset animals;
outputting a second question for inducing the user to select an animal that others think of as representing the user, from among the images of the plurality of preset animals;
receiving an input for selecting a second animal group of a second number from the user in response to the second question, the second animal group being used as objective determination data for determining personality of the user;
generating a characteristic pattern by combining characteristics of animals selected in the first animal group and the second animal group;
determining a user personality type based on the generated characteristic pattern and a personality type table; and
outputting the determined user personality type,
wherein the determining of the user personality type includes:
determining the user personality type as a pre-defined specific personality type that does not match symbolic personalities of the plurality of animals when the number of types selected most often by the user in each generated characteristic pattern is two or less, or the number of types selected most often by the user is four or less and the number of each of other types selected is only one without overlapping each other.

2. The method of claim 1, wherein each of the plurality of output animals corresponds to at least one of predetermined characteristics.

3. The method of claim 1, wherein the outputting of the images of the animals includes outputting the same number of animals for each predetermined characteristic on the screen.

4. The method of claim 1, wherein the generating of the characteristic pattern includes counting the number of times the animals are selected by the user for each group classified based on characteristics of the animals.

5. The method of claim 1, wherein the determining of the user personality type includes determining a personality type matching the classified group that is selected most often by the user as a main personality of the user, and determining a personality type matching the classified group that is selected second most often by the user as a secondary personality of the user.

6. The method of claim 1, wherein in the determining of the user personality type, the user personality type is effectively treated only when two or more characteristic types are selected by the user.

7. The method of claim 1, wherein in the determining of the user personality type, the user personality type is determined as a harmony type when the number of types selected most often by the user in each generated characteristic pattern is two or less, or the number of types selected most often by the user is four or less and the number of each of other types selected is only one without overlapping each other.

8. An apparatus for testing personality based on animal symbols, the apparatus comprising:
a processor configured to:
output images of a plurality of preset animals on a screen;
receive an input for selecting a first animal group of a first number from a user;
receive an input for selecting a second animal group of a second number from the user;
generate a characteristic pattern by combining characteristics of animals selected in the first animal group and the second animal group;
determine a user personality type based on the generated characteristic pattern and a personality type table; and
output the determined user personality type, and
a memory configured to store at least one of the images of the plurality of preset animals, the first animal group and the second animal group, the generated characteristic pattern, and the personality type table,
wherein the processor is configured to determine the user personality type as a pre-defined specific personality type that does not match symbolic personalities of the plurality of animals when the number of types selected most often by the user in each generated characteristic pattern is two or less, or the number of types selected most often by the user is four or less and the number of each of other types selected is only one without overlapping each other, in the determining of the user personality type.

9. The apparatus of claim 8, wherein each of the plurality of output animals corresponds to at least one of predetermined characteristics.

10. The apparatus of claim 8, wherein the processor outputs the same number of animals for each predetermined characteristic on the screen when the images of the animals are output.

11. The apparatus of claim 8, wherein the processor counts the number of times the animals are selected by the user for each group classified based on characteristics of the animals when the characteristic pattern is generated.

12. The apparatus of claim 8, wherein the processor determines a personality type matching the classified group that is selected most often by the user as a main personality of the user, and determines a personality type matching the classified group that is selected second most often by the user as a secondary personality of the user, in the determining of the user personality type.

13. The apparatus of claim 8, wherein the processor effectively treats the user personality type only when two or more characteristic types are selected by the user, in the determining of the user personality type.

14. The apparatus of claim 8, wherein the processor determines the user personality type as a harmony type when the number of types selected most often by the user in each generated characteristic pattern is two or less, or the number of types selected most often by the user is four or less and the number of each of other types selected is only one without overlapping each other, in the determining of the user personality type.

15. The method of claim 1, wherein in the determining of the user personality type, the user personality type is determined as a pre-defined personality type that matches when all types are represented in various ways, when the number of types selected most often by the user in each generated characteristic pattern is less than or equal to the first number, or the number of types selected most often by the user is less than or equal to the second number that is smaller than the first number and the number of each of other types selected is only one without overlapping each other.

16. The apparatus of claim 8, wherein the processor determines the user personality type as a pre-defined personality type that matches when all types are represented in various ways, when the number of types selected most often by the user in each generated characteristic pattern is less than or equal to the first number, or the number of types selected most often by the user is less than or equal to the second number that is smaller than the first number and the number of each of other types selected is only one without overlapping each other, in the determining of the user personality type.
